(19) United States
(12) Patent Application Publication
(10) Pub. No.: US 2026/0337613 A1
(43) Pub. Date: Oct. 29, 2026

(54) DOUBLE-GAP MODIFIED OLIGONUCLEOTIDES AND THEIR USE AGAINST HEPATITIS B AND D VIRUSES

(71) Applicant: ZHEJIANG PALO ALTO PHARMACEUTICALS, INC., Quzhou (CN)

(72) Inventors: Zhijian XI, Quzhou (CN); Chunping LU, Quzhou (CN)

(21) Appl. No.: 19/544,963

(22) Filed: Feb. 19, 2026

Related U.S. Application Data
(63) Continuation of application No. PCT/CN2024/111442, filed on Aug. 12, 2024.

(30) Foreign Application Priority Data
Aug. 22, 2023 (CN) 202311063767.4

(51) Int. Cl. C12N 15/113; A61P 31/20

(52) U.S. Cl. CPC C12N 15/1131; A61P 31/20; C12N 2310/11; C12N 2310/315; C12N 2310/321

(57) ABSTRACT

The present disclosure provides a compound, or a pharmaceutically acceptable salt, hydrate, or solvate thereof, wherein (a) the compound is a double-gap modified oligonucleotide; and wherein the oligonucleotide has a sequence as shown in SEQ ID NO.2, SEQ ID NO.3, SEQ ID NO.4, or SEQ ID NO.5; (b) the double-gap modified refers to each nucleoside moiety in the oligonucleotide being modified as shown in Formula I; and (c) internucleotide linkages of the oligonucleotide are unmodified or modified internucleotide linkages, wherein P1, P2, P3, S1, and S2 and specific modifications are as described in the specification. The double-gap modified oligonucleotides have significantly improved antiviral activity.

### CROSS-REFERENCE TO RELATED APPLICATIONS

This application is a continuation of International Application No. PCT/CN2024/111442 with a filling date of Aug. 12, 2024, designating the United states, now pending, and further claims to the benefit of priority from Chinese Application No. 202311063767.4 with a filing date of Aug. 22, 2023. The content of the aforementioned applications, including any intervening amendments thereto, are incorporated herein by reference.

### SEQUENCE LISTING INFORMATION

The contents of the electronic sequence listing (Sequence_Listing.xml; Size: 9,428 bytes; and Date of Creation: Jun. 30, 2026) is herein incorporated by reference in its entirety.

### TECHNICAL FIELD

The present disclosure belongs to the field of biomedicine, and specifically relates to double-gap modified oligonucleotides and their use against hepatitis B and D viruses.

### BACKGROUND

Hepatitis B is a viral disease caused by hepatitis B virus (HBV) infection, with main transmission routes including blood-borne, sexual, and mother-to-child transmission. The World Health Organization estimated that in 2015, over 200 million people worldwide were chronically infected with HBV, and 887,000 people died from complications caused by HBV infection. While 90% of adults infected with HBV can self-resolve, 90% of infants infected with HBV will develop chronic hepatitis. Chronic HBV infection may lead to liver fibrosis, further progressing to cirrhosis and hepatocellular carcinoma (HCC). Additionally, studies suggest that hepatitis B may increase the risk of pancreatic cancer.

Hepatitis D virus (HDV) is a satellite virus of HBV, relying on hepatitis B surface antigen (HBsAg) to form its complete, infectious HDV viral particle. HDV infection can only occur in patients concurrently infected with HBV. HDV/HBV co-infection complications are significant and also significantly increase the rate of progression from liver fibrosis to cirrhosis. For patients with chronic HDV infection, currently only interferon therapy is available as an intervention; there are no marketed drugs directly targeting the HDV virus. Existing treatment methods have poor efficacy and significant side effects.

HBV adheres to the surface of hepatocytes via low-affinity receptors, then enters hepatocytes via endocytosis aided by specific receptors on the hepatocyte membrane. The nucleocapsid disassembles and imports rcDNA into the host cell nucleus. Within the nucleus, rcDNA is converted into covalently closed circular DNA (cccDNA) via cellular DNA repair mechanisms. cccDNA serves as the storage form of HBV genetic material within the cell and is the main transcriptional template for HBV. The host cell recognizes promoters and enhancers on cccDNA to transcribe 3.5 kb, 2.4 kb, 2.1 kb, and 0.7 kb mRNAs, with the 3.5 kb being the pregenomic RNA (pgRNA). mRNAs enter the cytoplasm and translate into viral proteins, including core antigen (HBcAg), e antigen (HBeAg), surface antigen (HBsAg), x protein (HBx), and polymerase (Polymerase). Under the action of Polymerase, the HBV DNA minus strand is synthesized via reverse transcription using pgRNA as a template, then the plus strand is partially synthesized using the minus strand as a template, forming rcDNA. Concurrently, HBcAg assembles into nucleocapsids enveloping rcDNA to form viral core particles. After synthesis, HBsAg polymerizes in the endoplasmic reticulum and transports to the Golgi apparatus to package viral core particles. Assembled viral particles are finally secreted extracellularly via budding.

Following HBV infection of human hepatocytes, two main distinct particles are produced: one is the Dane particle, which is the complete HBV virus itself, comprising a viral nucleocapsid assembled from hepatitis B core antigen (HBcAg) and viral nucleic acid (RcDNA), and having a viral envelope composed of hepatitis B surface antigen (HBsAg); the other is subviral particles (SVP), which are non-infectious particles composed of lipids, cholesterol, cholesterol esters, and hepatitis B surface antigen (HBsAg). The hepatitis B surface antigen contained in SVPs constitutes the vast majority (>99.9%) of HBsAg in a patient's blood. HBV-infected hepatocytes also secrete an e-antigen (HBeAg) into the blood. Hepatitis B surface antigen (HBsAg), hepatitis B surface antibody (HBsAb), hepatitis B core antibody (HBcAb), hepatitis B e antigen (HBeAg), and hepatitis B e antibody (HBeAb) are important molecular markers for evaluating drug intervention on the virus.

The large amount of hepatitis B surface antigen (HBsAg) present in the blood of patients with chronic HBV infection, existing in the form of subviral particles (SVPs), can neutralize specific hepatitis B surface antibodies (HBsAb) secreted by B lymphocytes, leading to immune tolerance, while the minority HBV viral particles can evade immune surveillance. This may be one of the important reasons why HBV maintains chronic infection. Seroconversion of hepatitis B surface antigen (HBsAg) (clearance of HBsAg from the blood, appearance of free HBsAb) is a recognized prognostic indicator of functional control of viral infection during treatment. Another key reason for the characteristic persistence of chronic HBV infection is its stable circular DNA reservoir, HBV covalently closed circular DNA (cccDNA), synthesized in the nuclei of infected hepatocytes with the help of host DNA repair enzymes. cccDNA can stably persist in hepatocytes for a long time and can be continuously replenished. It can produce HBV viral nucleic acid RcDNA and mRNAs encoding all viral antigens via transcription and reverse transcription. Transcriptional inhibition or clearance of cccDNA is crucial for curing or functionally curing HBV infection. Long-term treatment with nucleoside (or nucleotide) analogs cannot completely eliminate cccDNA nor inhibit its transcription; therefore, hepatitis B surface antigen (HBsAg) expression levels are almost unaffected by nucleoside (or nucleotide) drugs. Immunomodulation can mediate humoral and cellular immunity, thereby inhibiting cccDNA transcription or clearing infected cells, but a large antigen load greatly inhibits this immune process. Therefore, significantly reducing antigens, especially hepatitis B surface antigen (HBsAg), combined with immunomodulation is an effective means to help patients achieve durable immune control.

Drugs clinically used to treat hepatitis B mainly include interferons and nucleoside (or nucleotide) analogs. Interferon drugs include conventional interferon and polyethylene glycol-modified long-acting interferons, the latter including Pegasys (PEG-IFNα-2a) and PegIntron (PEG-IFNα-2b). Nucleoside (or nucleotide) analogs include lamivudine, telbivudine, adefovir dipivoxil, tenofovir disoproxil fumarate (TDF), tenofovir alafenamide fumarate (TAF), entecavir, etc. These nucleoside analogs can effectively control viral replication and improve liver function, and thus are most widely used. Interferons require injection administration, have significant individual response variability, obvious adverse reactions, and poor efficacy. Nucleoside analogs only act on the replication process from pgRNA to rcDNA and have no inhibitory effect on other stages of the HBV life cycle. After long-term treatment, the HBeAg seroconversion rate remains low, and very few patients achieve HBsAg seroconversion. After 48 weeks of treatment with entecavir (354 cases) and tenofovir (176 cases), the HBsAg seroconversion rates in HBeAg-positive patients were 2% and 3.2%, respectively, and in HBeAg-negative patients were 0.3% and 0%, respectively. Since existing treatment regimens cannot cure hepatitis B, patients require long-term medication, potentially exposing them to significant side effects, such as nephrotoxicity and bone toxicity from long-term use of adefovir dipivoxil and tenofovir disoproxil fumarate. Existing drug therapies or combination therapies, except in a small fraction of patients (<3%), fail to elicit an effective immune response providing durable control of infection or functional cure, or HBsAg seroconversion. Curing or functionally curing chronic HBV infection is a huge unmet clinical need.

In summary, there is an urgent need in the field to discover and develop new antiviral treatment methods. Particularly, there is an urgent need for new therapies that can effectively inhibit hepatitis B viral antigens HBsAg and/or HBeAg and increase their seroconversion rates.

### SUMMARY

The object of the present disclosure is to provide novel compounds for anti-hepatitis B virus/hepatitis D virus therapy.

In a first aspect of the present disclosure, provided is a compound, or a pharmaceutically acceptable salt, hydrate, or solvate thereof, the compound is a double-gap modified oligonucleotide;
wherein:
(a) the oligonucleotide has a sequence as shown in SEQ ID NO.2, SEQ ID NO.3, SEQ ID NO.4, or SEQ ID NO.5;

| | |
|---|---|
| SEQ ID NO. 2 | GCAGAGGTGAAGCGAAGTGC |
| SEQ ID NO. 3 | GTGCAGAGGTGAAGCGAAGT |
| SEQ ID NO. 4 | AGAGGTGAAGCGAAGTGCAC |
| SEQ ID NO. 5 | GTGAAGCGAAGTGCACACGG |

(b) the double-gap modified refers to each nucleoside moiety in the oligonucleotide being modified as shown in Formula I;

P1-S1-P2-S2-P3 (I)

In Formula I,
P1 represents a modification region including at least 1 consecutive nucleotide at 5′-end;
P2 represents a modification region including at least 1 consecutive nucleotide starting from 3′-end of the S1 region;
P3 represents a modification region including at least 1 consecutive nucleotide at 3′-end;
S1 represents a spacer region including at least 1 consecutive nucleotide; and
S2 represents a spacer region including at least 1 consecutive nucleotide starting from 3′-end of the P2 region;
and,
in the modification region, all nucleoside moieties have sugar moiety modifications, and all nucleoside moieties optionally have base modifications; and
in the spacer region, all nucleoside moieties lack sugar moiety modifications, and all nucleoside moieties optionally have base modifications;
and,
(c) internucleotide linkages of the oligonucleotide are unmodified or modified internucleotide linkages; and, the modified internucleotide linkages refer to part or all of phosphodiester internucleotide linkages in the oligonucleotide each being independently replaced by an internucleotide linkage selected from the group consisting of: phosphorothioate internucleotide linkages, phosphorodithioate internucleotide linkages, or combinations thereof.

In another preferred embodiment, each nucleotide in P1, P2, and P3 is a modified nucleoside (preferably, the modified nucleoside comprises a modified sugar and a modified or unmodified base); and each nucleotide in S1 and S2 is an unmodified nucleoside or has an unmodified sugar.

In another preferred embodiment, the sugar moiety modification is selected from: 2′-O-methyl sugar moiety modification, 2′-O-methoxyethyl sugar moiety modification, or combinations thereof, preferably, it is 2′-O-methoxyethyl sugar moiety modification (moe).

In another preferred embodiment, the base modification is a 5-methyl modification of a cytosine (C) base (5-methyl, 5Me).

In another preferred embodiment, internucleotide linkages of the oligonucleotide are phosphorothioate internucleotide linkages, phosphorodithioate internucleotide linkages, or combinations thereof.

In another preferred embodiment, internucleotide linkages of the oligonucleotide are phosphorothioate linkages.

In another preferred embodiment, P1 represents a modification region including 2 to 6 (preferably 3 to 4) consecutive nucleotides at 5′-end.

In another preferred embodiment, P2 represents a modification region including 1 to 5 (preferably 1, 2, 3, or 4) consecutive nucleotides.

In another preferred embodiment, P3 represents a modification region including 2 to 6 (preferably 3 or 4) consecutive nucleotides.

In another preferred embodiment, S1 represents a spacer region including 3 to 7 (preferably 4, 5, 6, or 7, more preferably 4, 5, or 6, most preferably 5 or 6) consecutive nucleotides.

In another preferred embodiment, S2 represents a spacer region including 3 to 7 (preferably 4, 5, 6, or 7, more preferably 4, 5, or 6, most preferably 5 or 6) consecutive nucleotides.

In another preferred embodiment, (a) the oligonucleotide has a sequence as shown in SEQ ID NO.2, SEQ ID NO.3, SEQ ID NO.4, or SEQ ID NO.5; (b) the double-gap modified refers to each nucleoside moiety in the oligonucleotide being modified as shown in Formula I; P1-S1-P2-S2-P3 (I); in Formula I, P1 represents a modification region including 2 to 6 consecutive nucleotides at 5′-end; P2 represents a modification region including 1 to 5 consecutive nucleotides; P3 represents a modification region including 2 to 6 consecutive nucleotides; S1 represents a spacer region including 4, 5, or 6 consecutive nucleotides; S2 represents a spacer region including 4, 5, or 6 consecutive nucleotides; and, in the modification region, all nucleoside moieties have sugar moiety modifications, and all nucleoside moieties optionally have base modifications; and in the spacer region, all nucleoside moieties lack sugar moiety modifications, and all nucleoside moieties optionally have base modifications; wherein, the sugar moiety modification is selected from: 2′-O-methyl sugar moiety modification, 2′-O-methoxyethyl sugar moiety modification, or combinations thereof; the base modification is a 5-methyl modification of a cytosine (C) base; and, (c) internucleotide linkages of the oligonucleotide are modified internucleotide linkages; and, the modified internucleotide linkages refer to part or all of phosphodiester internucleotide linkages in the oligonucleotide each being independently replaced by an internucleotide linkage selected from the group consisting of: phosphorothioate internucleotide linkages, phosphorodithioate internucleotide linkages, or combinations thereof.

In another preferred embodiment, (a) the oligonucleotide has a sequence as shown in SEQ ID NO.2, SEQ ID NO.3, SEQ ID NO.4, or SEQ ID NO.5; (b) the double-gap modified refers to each nucleoside moiety in the oligonucleotide being modified as shown in Formula I; P1-S1-P2-S2-P3 (I); in Formula I, P1 represents a modification region including 3 to 4 consecutive nucleotides at 5′-end; P2 represents a modification region including 1, 2, 3, or 4 consecutive nucleotides; P3 represents a modification region including 3 or 4 consecutive nucleotides; S1 represents a spacer region including 5 or 6 consecutive nucleotides; S2 represents a spacer region including 5 or 6 consecutive nucleotides; and, in the modification region, all nucleoside moieties have sugar moiety modifications, and all nucleoside moieties optionally have base modifications; and in the spacer region, all nucleoside moieties lack sugar moiety modifications, and all nucleoside moieties optionally have base modifications; wherein, the sugar moiety modification is selected from: 2′-O-methyl sugar moiety modification, 2′-O-methoxyethyl sugar moiety modification, or combinations thereof, the base modification is a 5-methyl modification of a cytosine (C) base; and, (c) internucleotide linkages of the oligonucleotide are all phosphorothioate internucleotide linkages.

In another preferred embodiment, the oligonucleotide has a sequence as shown in SEQ ID NO.2 or SEQ ID NO.5.

In another preferred embodiment, the oligonucleotide has a sequence as shown in SEQ ID NO.2.

In another preferred embodiment, when the oligonucleotide has a sequence as shown in SEQ ID NO.2, the sugar moiety modification is 2′-O-methoxyethyl sugar moiety modification; and/or, when the oligonucleotide has a sequence as shown in SEQ ID NO.5, the sugar moiety modification is 2′-O-methyl sugar moiety modification.

In another preferred embodiment, the oligonucleotide has a sequence as shown in SEQ ID NO.2, and the sugar moiety modification is 2′-O-methoxyethyl sugar moiety modification.

In another preferred embodiment, (a) the oligonucleotide has a sequence as shown in SEQ ID NO.2; (b) the double-gap modified refers to each nucleoside moiety in the oligonucleotide being modified as shown in Formula I; P1-S1-P2-S2-P3 (I); in Formula I, P1 represents a modification region including 3 to 4 consecutive nucleotides at 5′-end; P2 represents a modification region including 1, 2, 3, or 4 consecutive nucleotides; P3 represents a modification region including 3 or 4 consecutive nucleotides; S1 represents a spacer region including 5 or 6 consecutive nucleotides; S2 represents a spacer region including 5 or 6 consecutive nucleotides; and, in the modification region, all nucleoside moieties have sugar moiety modifications, and all nucleoside moieties optionally have base modifications; and in the spacer region, all nucleoside moieties lack sugar moiety modifications, and all nucleoside moieties optionally have base modifications; wherein, the sugar moiety modification is 2′-O-methoxyethyl sugar moiety modification, or combinations thereof; the base modification is a 5-methyl modification of a cytosine (C) base; and, (c) internucleotide linkages of the oligonucleotide are all phosphorothioate internucleotide linkages.

In another preferred embodiment, the compound is selected from Table A and Table B:

TABLE A

| Oligo- | | |
|---|---|---|
| nucleotides | Sequence | SEQ ID |
| PA0070 | GTGAAGCGAAGTGCACACGG | SEQ ID NO. 5 |
| PA0071 | GTGCAGAGGTGAAGCGAAGT | SEQ ID NO. 3 |
| PA0073 | GTGAAGCGAAGTGCACACGG | SEQ ID NO. 5 |
| PA0074 | GTGCAGAGGTGAAGCGAAGT | SEQ ID NO. 3 |
| PA0075 | GTGAAGCGAAGTGCACACGG | SEQ ID NO. 5 |
| PA0076 | GTGCAGAGGTGAAGCGAAGT | SEQ ID NO. 3 |
| PA0077 | GTGAAGCGAAGTGCACACGG | SEQ ID NO. 5 |
| PA0078 | GTGCAGAGGTGAAGCGAAGT | SEQ ID NO. 3 |
| PA0079 | GTGAAGCGAAGTGCACACGG | SEQ ID NO. 5 |
| PA0080 | GTGCAGAGGTGAAGCGAAGT | SEQ ID NO. 3 |
| PA0081 | GTGAAGCGAAGTGCACACGG | SEQ ID NO. 5 |
| PA0082 | GTGCAGAGGTGAAGCGAAGT | SEQ ID NO. 3 |
| PA0083 | GTGAAGCGAAGTGCACACGG | SEQ ID NO. 5 |
| PA0084 | GTGCAGAGGTGAAGCGAAGT | SEQ ID NO. 3 |
| PA0085 | GTGAAGCGAAGTGCACACGG | SEQ ID NO. 5 |
| PA0086 | GTGCAGAGGTGAAGCGAAGT | SEQ ID NO. 3 |

In Table A, A/T/G/C represent conventional unmodified deoxyribonucleotide residues; A/T/G/C represent 2′-O-methyl modified nucleoside bases; and the internucleotide linkages of each oligonucleotide are all phosphorothioate internucleotide linkages;

TABLE B

| Oligo- | | |
|---|---|---|
| nucleotides | Sequence | SEQ ID |
| PA0088 | GCAGAGGTGAAGCGAAGTGC | SEQ ID NO. 2 |
| PA0089 | GCAGAGGTGAAGCGAAGTGC | SEQ ID NO. 2 |
| PA0114 | GCAGAGGTGAAGCGAAGTGC | SEQ ID NO. 2 |
| PA0090 | GCAGAGGTGAAGCGAAGTGC | SEQ ID NO. 2 |
| PA0115 | GCAGAGGTGAAGCGAAGTGC | SEQ ID NO. 2 |
| PA0091 | GCAGAGGTGAAGCGAAGTGC | SEQ ID NO. 2 |
| PA0116 | GCAGAGGTGAAGCGAAGTGC | SEQ ID NO. 2 |
| PA0092 | GCAGAGGTGAAGCGAAGTGC | SEQ ID NO. 2 |
| PA0093 | GCAGAGGTGAAGCGAAGTGC | SEQ ID NO. 2 |
| PA0094 | GCAGAGGTGAAGCGAAGTGC | SEQ ID NO. 2 |
| PA0095 | GCAGAGGTGAAGCGAAGTGC | SEQ ID NO. 2 |
| PA0096 | GTGCAGAGGTGAAGCGAAGT | SEQ ID NO. 3 |
| PA0097 | GTGCAGAGGTGAAGCGAAGT | SEQ ID NO. 3 |
| PA0098 | GTGCAGAGGTGAAGCGAAGT | SEQ ID NO. 3 |
| PA0099 | GTGCAGAGGTGAAGCGAAGT | SEQ ID NO. 3 |
| PA0100 | GTGCAGAGGTGAAGCGAAGT | SEQ ID NO. 3 |
| PA0101 | GTGCAGAGGTGAAGCGAAGT | SEQ ID NO. 3 |
| PA0102 | GTGCAGAGGTGAAGCGAAGT | SEQ ID NO. 3 |
| PA0103 | GTGCAGAGGTGAAGCGAAGT | SEQ ID NO. 3 |
| PA0104 | AGAGGTGAAGCGAAGTGCAC | SEQ ID NO. 4 |
| PA0105 | AGAGGTGAAGCGAAGTGCAC | SEQ ID NO. 4 |
| PA0106 | AGAGGTGAAGCGAAGTGCAC | SEQ ID NO. 4 |
| PA0107 | AGAGGTGAAGCGAAGTGCAC | SEQ ID NO. 4 |
| PA0108 | AGAGGTGAAGCGAAGTGCAC | SEQ ID NO. 4 |
| PA0109 | AGAGGTGAAGCGAAGTGCAC | SEQ ID NO. 4 |
| PA0110 | AGAGGTGAAGCGAAGTGCAC | SEQ ID NO. 4 |
| PA0111 | AGAGGTGAAGCGAAGTGCAC | SEQ ID NO. 4 |

In Table B, A/T/G represent conventional unmodified deoxyribonucleotide residues and C represents a 5-methyl modified deoxyribonucleotide residue; A/T/G/C represent 2′-O-methoxyethyl modified nucleoside bases; and the internucleotide linkages of each oligonucleotide are all phosphorothioate internucleotide linkages.

In another preferred embodiment, the compound is selected from: PA0088, PA0089, PA0114, PA0090, PA0115, PA0091, PA0116, PA0092, PA0093, PA0094, and PA0095.

In another preferred embodiment, the compound is selected from the group consisting of: 5′-Gmoe*^{5Me}Cmoe*Amoe*Gmoe*dA*dG*dG*dT*dG*Amoe*dA*dG*^{5M}edC*dG*dA*dA*G moe*Tmoe*Gmoe*^{5Me}Cmoe-3′ (PA0088); or
5′-Gmoe*^{5Me}Cmoe*Amoe*dG*dA*dG*dG*dT*dG*Amoe*dA*dG*^{5Me}dC*dG*dA*dA*dG*Tmoe*Gmoe*^{5Me}Cmoe-3′ (PA0089),
wherein, * represents phosphorothioate; moe in Amoe, Gmoe, ^{5Me}Cmoe and Tmoe represents a 2′-O-methoxyethyl sugar moiety modification on the corresponding nucleoside (i.e., Amoe, Gmoe, Cmoe and Tmoe represent 2′-O-methoxyethyl modified nucleosides); dA, dG, dC and dT in dA, dG, ^{5Me}dC, dT represent unmodified nucleosides; ^{5Me}dC in ^{5Med}C represents a 5-methyl base modification.

In a second aspect of the present disclosure, provided is a pharmaceutical composition, the pharmaceutical composition comprising a compound according to the first aspect, or a pharmaceutically acceptable salt, hydrate, or solvate thereof, and a pharmaceutically acceptable carrier.

In a third aspect of the present disclosure, provided is a use of a compound according to the first aspect, or a pharmaceutically acceptable salt, hydrate, or solvate thereof, or a pharmaceutical composition according to the second aspect, in the manufacture of a medicament for treating and/or preventing a disease associated with hepatitis B virus or hepatitis D virus.

In another preferred embodiment, the disease includes one or more diseases selected from: diseases associated with hepatitis B virus infection, diseases associated with hepatitis B virus and hepatitis D virus co-infection.

In another preferred embodiment, the disease may be an acute or chronic disease.

In another preferred embodiment, the disease includes viral hepatitis B, viral hepatitis D, liver fibrosis, cirrhosis, hepatocellular carcinoma (HCC), or combinations thereof.

In another preferred embodiment, the disease includes acute or chronic liver disease.

In a fourth aspect of the present disclosure, provided is a method for treating and/or preventing a disease associated with hepatitis B virus or hepatitis D virus, the method comprising: administering a safe and effective amount of a compound according to the first aspect, or a pharmaceutically acceptable salt, hydrate, or solvate thereof, or a pharmaceutical composition according to the second aspect, to a subject in need thereof.

In another preferred embodiment, the disease is as defined in the third aspect.

In another preferred embodiment, the method comprises administering a safe and effective amount of a compound according to the first aspect, or a pharmaceutically acceptable salt, hydrate, or solvate thereof, or a pharmaceutical composition according to the second aspect, to a subject in need thereof via intravenous injection and/or subcutaneous injection.

In another preferred embodiment, the subject is a mammal; preferably, the subject is selected from humans, rats, mice, or combinations thereof.

In a fifth aspect of the present disclosure, provided is a method for modulating HBV DNA/RNA, HBsAg and/or HBeAg expression, the method comprising: contacting a subject with a compound according to the first aspect, or a pharmaceutically acceptable salt, hydrate, or solvate thereof, thereby modulating HBV DNA/RNA, HBsAg and/or HBeAg expression.

In another preferred embodiment, the method is in vitro and non-therapeutic.

In another preferred embodiment, the subject is a cell.

In another preferred embodiment, the modulating refers to inhibiting the expression of HBV DNA/RNA, HBsAg and/or HBeAg, or reducing the level of HBV RNA and/or HBeAg extracellularly (e.g., in cell culture medium).

### BRIEF DESCRIPTION OF THE DRAWINGS

FIGS. 1 and 2 show the relative inhibition rates of HBsAg expression/secretion by antisense oligonucleotides with different gap modification patterns in HepG2.2.1.5 cells.

FIG. 3 shows the antiviral effects of antisense oligonucleotides with different gap modification patterns in the HepG2.2.15 cell line.

FIG. 4 shows the in vivo efficacy of gap-modified antisense oligonucleotides in animals.

FIG. 5 shows the body weight gain of animals administered gap-modified antisense oligonucleotides.

### DETAILED DESCRIPTION

After extensive and intensive research, the inventors unexpectedly discovered that modifying oligonucleotides with specific sequences (particularly the sequence shown in SEQ ID NO.2) in a double-gap manner can enhance, or even significantly enhance, the antiviral activity of these antisense oligonucleotides, as well as the concentration of HBsAg at the cellular level. Based on the above discovery, the inventors completed the present disclosure.

### Definitions

The term “oligonucleotide” refers to an oligomer of nucleotides from ribonucleic acid (RNA) and/or deoxyribonucleic acid (DNA). The term includes oligonucleotides composed of modified or unmodified nucleobases, modified or unmodified sugars (ribose or deoxyribose) and modified or unmodified internucleotide linkages (phosphodiesters), and wherein one or more bases in the oligonucleotides may optionally be replaced (e.g., T replaced by modified or unmodified U), as well as functionally similar oligonucleotides having non-naturally occurring moieties. In particular, modified or substituted oligonucleotides may be superior to natural forms due to desired properties such as, for example, reduced immunoreactivity, enhanced cellular uptake, enhanced affinity for nucleic acid targets, and/or increased stability to nuclease-mediated degradation. In the present disclosure, oligonucleotides may be single-stranded or double-stranded, including single-stranded molecules such as antisense oligonucleotides (ASOs), aptamers, miRNAs, etc., and double-stranded molecules such as small interfering RNA (siRNA) or small hairpin RNA (shRNA). Oligonucleotides may include various modifications, e.g., stabilizing modifications, and thus may have at least one modification or include at least one modifying group on phosphodiesters (partially or fully) and/or on the sugar and/or base. For example, oligonucleotides may undergo modifications including, but not limited to, one or more modifications, or may be fully modified to contain all the modified bonds or sugars or bases (that is to say, each phosphodiester, sugar, and base constituting the oligonucleotide is either unmodified, or partially modified or fully modified). In the present disclosure, modified internucleotide linkages may be phosphorothioate linkages and/or phosphorodithioate linkages. Other useful modifications for the present disclosure include, but are not limited to, modifications at the 2′-position of the sugar, including 2′-O-alkyl modifications (such as 2′-O-methyl modification, 2′-O-methoxyethyl modification (2′-MOE)), 2′-amino modification, 2′-halogen modifications (such as 2′-fluoro substitution); acyclic nucleotide analogs. Other 2′-position modifications are also well known in the art and may be used, such as locked nucleic acids. Specifically, oligonucleotides have modified bonds throughout or have each bond modified, e.g., phosphorothioate; have a 3′-cap and/or 5′-cap: including terminal 3′-5′ linkages. Base modifications may include 5′-methylation of cytosine bases (5′-methylcytosine) and/or 4′-thiolation of uracil bases (4′-thiouracil). When synthesis conditions are chemically compatible, different chemically compatible modified bonds may be combined, e.g., oligonucleotides having phosphorothioate bonds, 2′-ribose modifications (e.g., 2′-O-methylation) and modified bases (e.g., 5′-methylcytosine). Oligonucleotides may be further fully modified utilizing all these different modifications (e.g., each phosphorothioated bond, each 2′ modified ribose, and each modified base).

For brevity, unless otherwise specified, oligonucleotides represented in DNA/RNA sequence form or defined in a manner such as SEQ ID NO.2 herein include both modified and unmodified cases of oligonucleotides having that sequence.

The term “antisense oligonucleotide” refers to a single-stranded oligonucleotide having a nucleic acid base sequence that allows hybridization to a corresponding segment of a target nucleic acid.

As used herein, the term “nt” refers to nucleotide.

The term “complementary” refers to the ability of the nucleic acid base sequence of an antisense oligonucleotide to undergo accurate base pairing with the corresponding nucleic acid base sequence in a target nucleic acid, mediated by hydrogen bonding between corresponding nucleic bases, e.g., adenine base pairing with thymine (or uracil), guanine with cytosine.

In the present disclosure, the terms “comprising”, “including” or “containing” indicate that various components can be applied together in the mixture or composition of the present disclosure. Therefore, the terms “consisting essentially of . . . ” and “including . . . ” are encompassed within the term “comprising”.

In the present disclosure, the term “pharmaceutically acceptable” ingredient refers to a substance suitable for use in humans and/or animals without excessive adverse side reactions (such as toxicity, irritation, and allergic reactions), i.e., with a reasonable benefit/risk ratio.

In the present disclosure, the term “effective amount” refers to an amount of a therapeutic agent that treats, alleviates, or prevents a target disease or condition, or exhibits a detectable therapeutic or preventive effect. The precise effective amount for a subject depends on the subject's size and health, the nature and extent of the condition, and the chosen therapeutic agent and/or combination of therapeutic agents. Therefore, specifying an exact effective amount in advance is not useful. However, the effective amount can be determined by routine experimentation for a given condition, as can be judged by a clinician.

As used herein, the term “pharmaceutically acceptable salt” refers to a salt formed by a compound of the present disclosure with a base, suitable for use as a drug.

Unless otherwise specified, in the present disclosure, all occurrences of compounds are intended to include all possible optical isomers, such as a single chiral compound, or mixtures of various chiral compounds (i.e., racemates). Among all compounds of the present disclosure, each chiral carbon atom may optionally be R configuration or S configuration, or a mixture of R and S configurations.

Some compounds in the present disclosure may be crystallized or recrystallized with water or various organic solvents, in which case various solvates may form. Solvates of the present disclosure include stoichiometric solvates such as hydrates, etc., and also include compounds containing variable amounts of water when prepared by low-pressure lyophilization.

The present disclosure provides methods, compounds, and compositions for modulating HBV DNA/RNA and HBsAg, HBeAg expression. In the embodiments, compounds suitable for modulating HBV DNA/RNA and HBsAg, HBeAg expression are antisense oligonucleotides.

In certain embodiments, modulation may be performed in cells. In certain embodiments, modulation is performed in an animal body. In certain embodiments, the animal is human. In certain embodiments, HBV RNA levels are reduced. In certain embodiments, HBV-DNA levels are reduced. In certain embodiments, HBsAg levels are reduced. In certain embodiments, HBeAg levels are reduced. The reduction occurs in a dose- and time-dependent manner.

Also provided are methods, compounds, and compositions suitable for preventing, treating, and ameliorating diseases, disorders, and conditions. In certain embodiments, the HBV-associated diseases, disorders, and conditions are acute or chronic liver diseases. In certain embodiments, the liver diseases, disorders, and conditions include viral hepatitis B, viral hepatitis D, liver fibrosis, cirrhosis, hepatocellular carcinoma (HCC), etc.

In certain embodiments, the treatment methods include administering HBV antisense oligonucleotides to an individual in need by intravenous injection or subcutaneous injection.

### Oligonucleotides (Antisense Oligonucleotides)

The present disclosure unexpectedly discovered oligonucleotides (or referred to as antisense oligonucleotides) capable of inhibiting hepatitis B viral DNA/RNA as well as viral protein expression such as hepatitis B virus (HBV) surface antigen (HBsAg) and e antigen (HBeAg). Typically, the antisense oligonucleotides provided by the present disclosure are complementary to a pan-genotypic conserved sequence segment of the hepatitis B virus genome. For example, the antisense oligonucleotides provided by the present disclosure are complementary to, e.g., a pan-genotypic conserved sequence segment as shown in SEQ ID NO.6 within the hepatitis B virus genome (genotype D) shown in SEQ ID NO.1, or sequences having greater than 96%, greater than 98% homology with the sequence.

| | |
|---|---|
| SEQ ID | TTCCACAACC TTCCACCAAA CTCTGCAAGA TCCCAGAGTG |
| NO. 1 | AGAGGCCTGT |
| | ATTTCCCTGC TGGTGGCTCC AGTTCAGGAA CAGTAAACCC |
| | TGTTCTGACT |
| | ACTGCCTCTC CCTTATCGTC AATCTTCTCG AGGATTGGGG |
| | ACCCTGCGCT |
| | GAACATGGAG AACATCACAT CAGGATTCCT AGGACCCCTT |
| | CTCGTGTTAC |
| | AGGCGGGGTT TTTCTTGTTG ACAAGAATCC TCACAATACC |
| | GCAGAGTCTA |
| | GACTCGTGGT GGACTTCTCT CAATTTTCTA GGGGGAACTA |
| | CCGTGTGTCT |
| | TGGCCAAAAT TCGCAGTCCC CAACCTCCAA TCACTCACCA |
| | ACCTCTTGTC |
| | CTCCAACTTG TCCTGGTTAT CGCTGGATGT GTCTGCGGCG |
| | TTTTATCATC |
| | TTCCTCTTCA TCCTGCTGCT ATGCCTCATC TTCTTGTTGG |
| | TTCTTCTGGA |
| | CTATCAAGGT ATGTTGCCCG TTTGTCCTCT AATTCCAGGA |
| | TCCTCAACAA |
| | CCAGCACGGG ACCATGCCGG ACCTGCATGA CTACTGCTCA |
| | AGGAACCTCT |
| | ATGTATCCCT CCTGTTGCTG TACCAAACCT TCGGACGGAA |
| | ATTGCACCTG CCATCATCCT GGGCTTTCGG AAAATTCCTA |
| | TATTCCCATC |
| | TGGGAGTGGG |
| | CCTCAGCCCG TTTCTCCTGG CTCAGTTTAC TAGTGCCATT |
| | TGTTCAGTGG |
| | TTCGTAGGGC TTTCCCCCAC TGTTTGGCTT TCAGTTATAT |
| | GGATGATGTG |
| | GTATTGGGGG CCAAGTCTGT ACAGCATCTT GAGTCCCTTT |
| | TTACCGCTGT |
| | TACCAATTTT CTTTTGTCTT TGGGTATACA TTTAAACCCT |
| | AACAAAACAA |
| | AGAGATGGGG TTACTCTCTA AATTTTATGG GTTATGTCAT |
| | TGGATGTTAT |
| | GGGTCCTTGC CACAAGAACA CATCATACAA AAAATCAAAG |
| | AATGTTTTAG |
| | AAAACTTCCT ATTAACAGGC CTATTGATTG GAAAGTATGT |
| | CAACGAATTG |
| | TGGGTCTTTT GGGTTTTGCT GCCCCTTTTA CACAATGTGG |
| | TTATCCTGCG |
| | TTGATGCCTT TGTATGCATG TATTCAATCT AAGCAGGCTT |
| | TCACTTTCTC |
| | GCCAACTTAC AAGGCCTTTC TGTGTAAACA ATACCTGAAC |
| | CTTTACCCCG |
| | TTGCCCGGCA ACGGCCAGGT CTGTGCCAAG TGTTTGCTGA |
| | CGCAACCCCC |
| | ACTGGCTGGG GCTTGGTCAT GGGCCATCAG CGCATGCGTG |
| | GAACCTTTTC |
| | GGCTCCTCTG CCGATCCATA CTGCGGAACT CCTAGCCGCT |
| | TGTTTTGCTC |
| | GCAGCAGGTC TGGAGCAAAC ATTATCGGGA CTGATAACTC |
| | TGTTGTCCTA |
| | TCCCGCAAAT ATACATCGTT TCCATGGCTG CTAGGCTGTG |
| | CTGCCAACTG |
| | GATCCTGCGC GGGACGTCCT TTGTTTACGT CCCGTCGGCG |
| | CTGAATCCTG |
| | CGGACGACCC TTCTCGGGGT CGCTTGGGAC TCTCTCGTCC |
| | CCTTCTCCGT |
| | CTGCCGTTCC GACCGACCAC GGGGCGCACC TCTCTTTACG |
| | CGGACTCCCC |
| | GTCTGTGCCT TCTCATCTGC CGGACCGTGT GCACTTCGCT |
| | TCACCTCTGC |
| | ACGTCGCATG GAGACCACCG TGAACGCCCA CCAAATATTG |
| | CCCAAGGTCT |
| | TACATAAGAG GACTCTTGGA CTCTCAGCAA TGTCAACGAC |
| | CGACCTTGAG |
| | GCATACTTCA AAGACTGTTT GTTTAAAGAC TGGGAGGAGT |
| | TGGGGGAGGA |
| | GATTAGGTTA AAGGTCTTTG TACTAGGAGG CTGTAGGCAT |
| | AAATTGGTCT |
| | GCGCACCAGC ACCATGCAAC TTTTTCACCT CTGCCTAATC |
| | ATCTCTTGTT TGTTCAAGCC TCCAAGCTGT GCCTTGGGTG |
| | CATGTCCTAC |
| | GCTTTGGGGC |
| | ATGGACATCG ACCCTTATAA AGAATTTGGA GCTACTGTGG |
| | AGTTACTCTC |
| | GTTTTTGCCT TCTGACTTCT TTCCTTCAGT ACGAGATCTT |
| | CTAGATACCG |
| | CCTCAGCTCT GTATCGGGAA GCCTTAGAGT CTCCTGAGCA |
| | TTGTTCACCT |
| | CACCATACTG CACTCAGGCA AGCAATTCTT TGCTGGGGGG |
| | AACTAATGAC |
| | TCTAGCTACC TGGGTGGGTG TTAATTTGGA AGATCCAGCG |
| | TCTAGAGACC |
| | TAGTAGTCAG TTATGTCAAC ACTAATATGG GCCTAAAGTT |
| | CAGGCAACTC |
| | TTGTGGTTTC ACATTTCTTG TCTCACTTTT GGAAGAGAAA |
| | CAGTTATAGA |
| | GTATTTGGTG TCTTTCGGAG TGTGGATTCG CACTCCTCCA |
| | GCTTATAGAC |
| | CACCAAATGC CCCTATCCTA TCAACACTTC CGGAGACTAC |
| | TGTTGTTAGA |
| | CGACGAGGCA GGTCCCCTAG AAGAAGAACT CCCTCGCCTC |
| | GCAGACGAAG |
| | GTCTCAATCG CCGCGTCGCA GAAGATCTCA ATCTCGGGAA |
| | TCTCAATGTT |
| | AGTATTCCTT GGACTCATAA GGTGGGGAAC TTTACTGGGC |
| | TTTATTCTTC |
| | TACTGTACCT GTCTTTAATC CTCATTGGAA AACACCATCT |
| | TTTCCTAATA |
| | TACATTTACA CCAAGACATT ATCAAAAAAT GTGAACAGTT |
| | TGTAGGCCCA |
| | CTCACAGTTA ATGAGAAAAG AAGATTGCAA TTGATTATGC |
| | CTGCCAGGTT |
| | TTATCCAAAG GTTACCAAAT ATTTACCATT GGATAAGGGT |
| | ATTAAACCTT |
| | ATTATCCAGA ACATCTAGTT AATCATTACT TCCAAACTAG |
| | ACACTATTTA |
| | CACACTCTAT GGAAGGCGGG TATATTATAT AAGAGAGAAA |
| | CAACACATAG |
| | CGCCTCATTT TGTGGGTCAC CATATTCTTG GGAACAAGAT |
| | CTACAGCATG |
| | GGGCAGAATC TTTCCACCAG CAATCCTCTG GGATTCTTTC |
| | CCGACCACCA |
| | GTTGGATCCA GCCTTCAGAG CAAACACCGC AAATCCAGAT |
| | TGGGACTTCA |
| | ATCCCAACAA GGACACCTGG CCAGACGCCA ACAAGGTAGG |
| | AGCTGGAGCA |
| | TTCGGGCTGG GTTTCACCCC ACCGCACGGA GGCCTTTTGG |
| | GGTGGAGCCC |
| | TCAGGCTCAG GGCATACTAC AAACTTTGCC AGCAAATCCG |
| | CCTCCTGCCT |
| | CCACCAATCG CCAGTCAGGA AGGCAGCCTA CCCCGCTGTC |
| | TCCACCTTTG |
| | AGAAACACTC ATCCTCAGGC CATGCAGTGG AA |
| SEQ ID | CTGCCGGACCGTGTGCACTTCGCTTCACCTCTGCACGTCGCA |
| NO. 6 | TGGA |

Preferably, the sequence of the oligonucleotide of the present disclosure is as shown in SEQ ID NO.2, SEQ ID NO.3, SEQ ID NO.4, and SEQ ID NO.5, more preferably as shown in SEQ ID NO.2.

In another preferred embodiment, the oligonucleotide is complementary to at least a portion of the fragment in SEQ ID NO.6.

In another preferred embodiment, the oligonucleotide is complementary to at least a portion of SEQ ID NO.6 and simultaneously complementary to at least a portion of SEQ ID NO.1.

In another preferred embodiment, the oligonucleotide is at least 96% complementary to SEQ ID NO.6.

### Double-Gap Modified Oligonucleotides

The present disclosure also provides modified antisense oligonucleotides. The preferred antisense oligonucleotides provided by the present disclosure, through double-gap design, i.e., modification of the ribose of nucleotides at the 5′ and 3′ ends of the oligonucleotide, improve affinity for target RNA, enhance efficacy, may reduce effective dose, and increase the safety window; simultaneously, the ribose modifications at both ends further improve oligonucleotide stability. A more preferred modified antisense oligonucleotide provided by the present disclosure is a phosphorothioate modified oligonucleotide, wherein the phosphorothioate modification improves in vivo stability of the oligonucleotide, enhances plasma protein binding, and facilitates distribution of the oligonucleotide to target tissues such as liver. The most preferred antisense oligonucleotide provided by the present disclosure is a double-gap antisense oligonucleotide, wherein the ribose moieties of nucleotides at the 5′ and 3′ ends and in the middle of the sequence are modified, and the base of cytosine nucleotides in CpG sequences (i.e., cytosine (C)-phosphate (p)-guanine (G)) is modified, thereby further improving oligonucleotide affinity and reducing off-target toxicity due to activation of innate immune Toll-like receptors.

In one specific embodiment, provided is a compound, or a pharmaceutically acceptable salt, hydrate, or solvate thereof, wherein the compound is a modified or unmodified oligonucleotide, and the length of the oligonucleotide is 14-26 nt (e.g., 16-24 nt, preferably, 18-22 nt, most preferably, 20 nt); and, the oligonucleotide has the structural feature of Formula I:

P1-S1-P2-S2-P3 (Formula I)
wherein, P1, P2, and P3 are one or more modified nucleotides, S1 and S2 are deoxyribonucleosides, the spacer region represented by S1 is Gap1, and the spacer region represented by S2 is Gap2.

The modification is one or more modifications selected from the group consisting of:
(i) nucleoside modification; the nucleoside modification includes 2′-O-methyl sugar moiety modification, 2′-O-methoxyethyl sugar moiety modification, and/or 5-methyl modification of cytosine;
(ii) internucleotide linkage modification; the internucleotide linkage modification refers to part or all of the internucleotide linkages in the oligonucleotide being replaced by phosphorothioate internucleotide linkages and/or phosphorodithioate internucleotide linkages.

In another preferred embodiment, each nucleotide in P1, P2, and P3 is a modified nucleoside (preferably, the modified nucleoside comprises a modified sugar and a modified or unmodified base);
each nucleotide in S1 and S2 is an unmodified nucleoside or has an unmodified sugar;
the various nucleoside moieties of the oligonucleotide are each independently connected via phosphodiesters or phosphorothioate bonds (preferably, all connected via phosphorothioate bonds).

In another preferred embodiment, at least part of the internucleotide linkages in the modified oligonucleotide are modified internucleotide linkages (i.e., phosphorothioate internucleotide linkages).

In another preferred embodiment, all internucleotide linkages in the modified oligonucleotide are modified internucleotide linkages (i.e., phosphorothioate internucleotide linkages).

In another preferred embodiment, the P1 corresponds to a region of 2-6 nt at 5′-end where modification is present.

In another preferred embodiment, the S1 corresponds to a region in the spacer region Gap1 comprising L₁ consecutive nucleotides without nucleoside modification, wherein L₁ is a positive integer from 3-7, preferably 4, 5, or 6, more preferably 5 or 6.

In another preferred embodiment, the P2 corresponds to a region of 2-5 nt where modification is present.

In another preferred embodiment, the S2 corresponds to a region in the spacer region Gap2 comprising L₂ consecutive nucleotides without nucleoside modification, wherein L₂ is a positive integer from 3-7, preferably 4, 5, or 6, more preferably 5 or 6.

In another preferred embodiment, the P3 corresponds to a region of 2-6 nt at 3′-end.

In another preferred embodiment, the length of P1 is 3, 4, or 5 nt, the length of S1 is 4, 5, or 6 nt, the length of P2 is 2, 3, or 4 nt, the length of S2 is 4, 5, or 6 nt, and/or the length of P3 is 3, 4, or 5 nt.

In another preferred embodiment, in P1, P2, and P3, the modified nucleosides are each independently nucleosides with modified sugar moieties, or nucleosides with both sugar moiety and base modified.

In another preferred embodiment, the nucleoside with modified sugar moiety refers to a nucleoside whose sugar contains a 2′-O-methyl group or a 2′-O-methoxyethyl group.

In another preferred embodiment, the nucleoside with both sugar moiety and base modified refers to a nucleoside whose sugar contains a 2′-O-methyl group or a 2′-O-methoxyethyl group and whose base is 5-methylcytosine.

In another preferred embodiment, in P1, P2, and P3, the modified nucleosides are each independently nucleosides with modified sugar moieties, or nucleoside moieties with both sugar moiety and base modified when the base is cytosine;
wherein, the nucleoside with modified sugar moiety refers to a nucleoside whose sugar contains a 2′-O-methyl group or a 2′-O-methoxyethyl group, and the nucleoside with both sugar moiety and base modified refers to a nucleoside whose sugar contains a 2′-O-methyl group or a 2′-O-methoxyethyl group and whose base is 5-methylcytosine.

In another specific embodiment, the present disclosure provides an oligonucleotide, or an optical isomer, pharmaceutically acceptable salt, hydrate, or solvate thereof, the antisense oligonucleotide sequence being selected from: SEQ ID NO. 2-5, and wherein one or more T may optionally be replaced by U.

| | |
|---|---|
| SEQ ID NO. 2 | GCAGAGGTGAAGCGAAGTGC |
| SEQ ID NO. 3 | GTGCAGAGGTGAAGCGAAGT |
| SEQ ID NO. 4 | AGAGGTGAAGCGAAGTGCAC |
| SEQ ID NO. 5 | GTGAAGCGAAGTGCACACGG |

In another preferred embodiment, the modified oligonucleotide is PA0088, its sequence is SEQ ID No.2, with sugar and base modifications as: 5′-Gmoe*^{5Me}Cmoe*Amoe*Gmoe*dA*dG*dG*dT*dG*Amoe*dA*dG*^{5Me}dC*dG*dA*dA*G moe*Tmoe*Gmoe*^{5Me}Cmoe-3′, wherein *: phosphorothioate; Amoe, Gmoe, ^{5Me}Cmoe, Tmoe: 2′-O-methoxyethyl modified monomers; dA, dG, ^{5Me}dC, dT: natural DNA monomers; ^{5Me}dC: 5-methyl base modification.

In another preferred embodiment, the modified oligonucleotide is PA0089, its sequence is SEQ ID No.2, with sugar and base modifications as: 5′-Gmoe*^{5Me}Cmoe*Amoe*dG*dA*dG*dG*dT*dG*Amoe*dA*dG*^{5Me}dC*dG*dA*dA*dG* Tmoe*Gmoe*^{5Me}Cmoe-3′, wherein *: phosphorothioate; Amoe, Gmoe, ^{5Me}Cmoe, Tmoe: 2′-O-methoxyethyl modified monomers; dA, dG, ^{5Me}dC, dT: natural DNA monomers; ^{5Me}: 5-methyl base modification.

### Preparation of Antisense Oligonucleotides

The antisense oligonucleotides of the present disclosure can be prepared and synthesized using conventional synthesis methods in the oligonucleotide industry. For example, phosphorothioate linkages can be synthesized using standard phosphoramidite chemistry on equipment such as the GE OP100, using 1,2-Benzodithiol-3-one-1,1-dioxide as an oxidizing agent instead of iodine.

### Pharmaceutical Compositions and Administration Methods

Since the compounds of the present disclosure (or the modified or unmodified oligonucleotides of the present disclosure) have excellent ability to inhibit hepatitis B viral DNA replication, the compounds of the present disclosure and isomers thereof (e.g., optical isomers), crystalline forms, solvates, pharmaceutically acceptable inorganic salts, and pharmaceutical compositions containing the compounds as the main active ingredient can be used for treating, preventing, and alleviating diseases associated with or caused by hepatitis B virus (i.e., hepatitis B virus) infection or diseases associated with or caused by hepatitis B virus and hepatitis D virus co-infection. These diseases may be acute or chronic. Based on existing technology, the compounds of the present disclosure can be used to treat the following diseases: hepatitis B; pancreatic cancer, cirrhosis, hepatocellular carcinoma, etc. (e.g., pancreatic cancer, cirrhosis, and hepatocellular carcinoma caused by chronic hepatitis B).

The pharmaceutical composition provided by the present disclosure comprises a compound of the present disclosure or other pharmacologically acceptable forms thereof (such as optical isomers, pharmaceutically acceptable salts, hydrates, or solvates) within a safe and effective amount range, and pharmaceutically acceptable auxiliaries, diluents, or carriers. Herein, “safe and effective amount” refers to an amount of the compound sufficient to significantly improve the condition, without producing serious side effects. Typically, the pharmaceutical composition contains 1-2000 mg of the compound of the present disclosure per dose, more preferably, contains 10-500 mg of the compound of the present disclosure per dose. Preferably, the “one dose” is one ampoule or vial.

“Pharmaceutically acceptable carrier” refers to one or more compatible solid or liquid filler or gel substances, which are suitable for human use, and must have sufficient purity and sufficiently low toxicity. “Compatible” herein means that the components in the composition can be blended with the compound of the present disclosure and with each other without significantly reducing the efficacy of the compound. Examples of pharmaceutically acceptable carriers include cellulose and its derivatives (e.g., sodium carboxymethyl cellulose, sodium ethyl cellulose, cellulose acetate, etc.), gelatin, talc, solid lubricants (e.g., stearic acid, magnesium stearate), calcium sulfate, vegetable oils (e.g., soybean oil, sesame oil, peanut oil, olive oil, etc.), polyols (e.g., propylene glycol, glycerol, mannitol, sorbitol, etc.), emulsifiers (e.g., Tween®), wetting agents (e.g., sodium lauryl sulfate), colorants, flavoring agents, stabilizers, antioxidants, preservatives, pyrogen-free water, etc.

### Administration Methods

The compounds and compositions containing them of the present disclosure can be administered by any suitable means, e.g., oral intake; oral inhalation; via subcutaneous, intravenous injection or infusion. The compounds or compositions containing them of the present disclosure may be administered in the form of dosage unit formulations containing non-toxic pharmaceutically acceptable carriers or diluents; or may be in the form of immediate-release or sustained-release formulations.

Indicative effective dosing regimens for agents based on the antisense oligonucleotides of the present disclosure for administration to humans, dosing regimens typically used for other antisense oligonucleotides, and the conventional use of weekly parenteral administration of 100-500 mg of compound established in the art are used.

Based on the disclosure presented herein, treatment of a subject having HBV infection or HBV/HDV co-infection with pharmaceutically acceptable antisense oligonucleotide formulations is useful.

Main advantages of the present disclosure include:
(a) The double-gap modified compounds or oligonucleotides of the present disclosure have significantly improved antiviral capability compared to unmodified or non-double-gap modified oligonucleotides.
(b) The compounds or oligonucleotides provided by the present disclosure can effectively inhibit viral gene products at the transcriptional level in vitro, thereby significantly inhibiting hepatitis B viral antigens (e.g., HBsAg).
(c) The modified oligonucleotides provided by the present disclosure, while enhancing in vivo stability through modification, have comparable or even significantly superior ability to inhibit hepatitis B viral antigens (e.g., HBsAg) compared to unmodified oligonucleotides.

In the following examples, experimental methods without specified conditions are generally performed under conventional conditions, for example, as described in Sambrook et al., Molecular Cloning: A Laboratory Manual (New York: Cold Spring Harbor Laboratory Press, 1989), or as recommended by the manufacturer. Unless otherwise stated, percentages and parts are by weight percentage and weight parts.

It should be understood that the skilled in the art can obtain the oligonucleotides used in the examples based on the sequences and corresponding modifications described in the examples, according to conventional techniques in the art (such as standard solid-phase synthesis methods) and using commercially available or synthesized raw materials (such as modified or unmodified nucleosides) prepared according to known methods.

### Example 1 Antiviral Effects of Antisense Oligonucleotides with Different Gap

### Modification Patterns in HepG2.2.15 Cell Line

The HepG2.2.1.5 cell line stably expresses and replicates HBV virus and secretes HBV viral particles, HBsAg, and HBeAg into the cell supernatant. HepG2.2.1.5 cells were cultured in DMEM: F12|medium (Hyclone) containing 10% FBS (ExCell Bio) and 400 μg/ml G418, and used after three passages.

Day 0: siRNAs were diluted in PBS to two concentrations (final concentrations 1 nM and 10 nM). HepG2.2.15 cells were taken, washed with DPBS, digested with trypsin, adjusted to appropriate density with 10% FBS DMEM: F12|medium (without G418). Cells were seeded and simultaneously transfected with siRNA using Lipofectamine RNAiMax. Cells were seeded at a density of 225,000 cells per well into a 96-well plate, with 135 μl culture medium per well. Cells were incubated in a 5% CO₂, 37° C. incubator for 3 days. Collected cell supernatants were assayed for HBsAg by ELISA. Simultaneously, cell viability was detected by CellTiter-Glo.

ELISA is briefly described as follows: 50 μl of sample and standard were added to the reaction plate wells, respectively, then 50 μl of enzyme conjugate was added to each well, shaken to mix, incubated at 37° C. for 60 minutes, then washed 5 times with wash buffer, then 50 μl of luminescent substrate was added to each well, mixed, reacted at room temperature while protected from light for 10 minutes, and finally chemiluminescence intensity was detected with a microplate reader.

CellTiter-Glo is briefly described as follows: 100 μl of CTG was added per well, reacted while protected from light for 10 min. Chemiluminescence intensity was detected with a microplate reader.

The antisense oligonucleotides used are described in Table 1.

TABLE 1
Description of oligonucleotides used in Example 1

| | | | P1-S1-P2-S2-P3 |
|---|---|---|---|
| | | | in Gap |
| Oligo- | | | modification |
| nucleotides | Sequence | SEQ ID | patterns |
| PA0070 | GTGAAGCGAAGTGCACACGG | SEQ ID NO. 5 | 4-5-1-6-4 |
| PA0071 | GTGCAGAGGTGAAGCGAAGT | SEQ ID NO. 3 | 4-5-1-6-4 |
| PA0073 | GTGAAGCGAAGTGCACACGG | SEQ ID NO. 5 | 3-6-1-7-3 |
| PA0074 | GTGCAGAGGTGAAGCGAAGT | SEQ ID NO. 3 | 3-6-1-7-3 |
| PA0075 | GTGAAGCGAAGTGCACACGG | SEQ ID NO. 5 | 4-5-2-5-4 |
| PA0076 | GTGCAGAGGTGAAGCGAAGT | SEQ ID NO. 3 | 4-5-2-5-4 |
| PA0077 | GTGAAGCGAAGTGCACACGG | SEQ ID NO. 5 | 3-6-2-6-3 |
| PA0078 | GTGCAGAGGTGAAGCGAAGT | SEQ ID NO. 3 | 3-6-2-6-3 |
| PA0079 | GTGAAGCGAAGTGCACACGG | SEQ ID NO. 5 | 4-4-3-5-4 |
| PA0080 | GTGCAGAGGTGAAGCGAAGT | SEQ ID NO. 3 | 4-4-3-5-4 |
| PA0081 | GTGAAGCGAAGTGCACACGG | SEQ ID NO. 5 | 3-5-3-6-3 |
| PA0082 | GTGCAGAGGTGAAGCGAAGT | SEQ ID NO. 3 | 3-5-3-6-3 |
| PA0083 | GTGAAGCGAAGTGCACACGG | SEQ ID NO. 5 | 4-4-4-4-4 |
| PA0084 | GTGCAGAGGTGAAGCGAAGT | SEQ ID NO. 3 | 4-4-4-4-4 |
| PA0085 | GTGAAGCGAAGTGCACACGG | SEQ ID NO. 5 | 3-5-4-5-3 |
| PA0086 | GTGCAGAGGTGAAGCGAAGT | SEQ ID NO. 3 | 3-5-4-5-3 |
| PA0087 | GTGAAGCGAAGTGCACACGG | SEQ ID NO. 5 | 5-10-5 |

A/T/G/C represent conventional unmodified deoxyribonucleotide residues; A/T/G/C represent 2′-O-methyl modified nucleoside bases; and the internucleotide linkages of each oligonucleotide are all phosphorothioate internucleotide linkages.

The formula for calculating the relative inhibition rate of HBsAg expression/secretion by transfected antisense oligonucleotides in HepG2.2.1.5 cells is:

Relative Inhibition Rate=100%*[1−(HBsAg concentration of treatment group/HBsAg concentration of blank control group)]

The relative inhibition rates for each antisense oligonucleotide treatment group are shown in Table 2.

TABLE 2
Relative inhibition rate of HBsAg expression/secretion
by oligonucleotides in Example 1 in HepG2.2.1.5 cells

HBsAg Relative inhibition rate (%)
Average of Two Experiments

| Oligonucleotides | 1 nM | 10 nM |
|---|---|---|
| PA0070 | 6.80 ± 0.94 | 88.97 ± 1.78 |
| PA0071 | 2.16 ± 0.36 | 54.60 ± 11.88 |
| PA0073 | 7.97 ± 3.28 | 86.24 ± 2.21 |
| PA0074 | 11.99 ± 3.59 | 51.84 ± 4.62 |
| PA0075 | 22.52 ± 3.02 | 90.33 ± 1.57 |
| PA0076 | 10.34 ± 2.81 | 63.72 ± 7.55 |
| PA0077 | 14.01 ± 3.56 | 90.94 ± 1.14 |
| PA0078 | 9.17 ± 4.56 | 66.48 ± 4.73 |
| PA0079 | 18.28 ± 1.69 | 91.62 ± 1.15 |
| PA0080 | 5.37 ± 2.07 | 67.75 ± 1.43 |
| PA0081 | 22.88 ± 2.81 | 95.73 ± 0.31 |
| PA0082 | 9.00 ± 1.07 | 78.55 ± 3.25 |
| PA0083 | 7.37 ± 1.04 | 82.09 ± 2.44 |
| PA0084 | 6.82 ± 5.78 | 56.44 ± 7.68 |
| PA0085 | 12.61 ± 1.53 | 84.50 ± 2.29 |
| PA0086 | 8.36 ± 1.79 | 68.73 ± 2.98 |
| PA0087 | 8.55 ± 0.40 | 80.24 ± 1.09 |

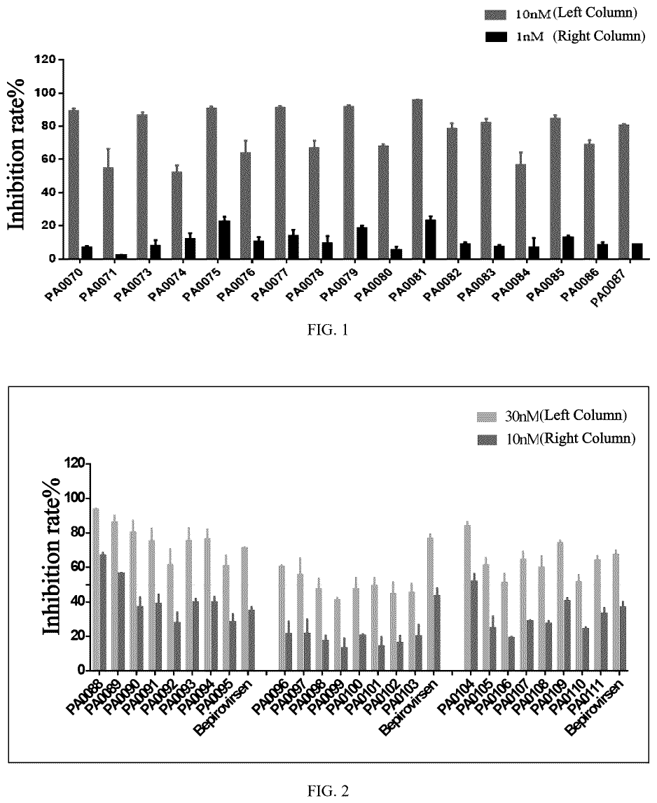

From Table 2 and FIG. 1, it can be seen that compared to the single-gap modified antisense oligonucleotide PA0087, adding double-gap modification helps improve the relative inhibition rate of HBsAg. Comprehensive results from the two experiments show that the weighted average of PA0075, PA0077, PA0079, and PA0081 is significantly better than that of the single-gap modified antisense oligonucleotide.

### Example 2 Antiviral Effects of Antisense Oligonucleotides with Different Gap Modification Patterns in HepG2.2.15 Cell Line

The HepG2.2.1.5 cell line stably expresses and replicates HBV virus and secretes HBV viral particles, HBsAg, and HBeAg into the cell supernatant. HepG2.2.1.5 cells were cultured in DMEM: F12|medium (Hyclone) containing 10% FBS (ExCell Bio) and 400 μg/ml G418, and used after three passages.

Day 0: siRNAs were diluted in PBS to two concentrations (final concentrations 10 nM and 30 nM). HepG2.2.15 cells were taken, washed with DPBS, digested with trypsin, adjusted to appropriate density with 10% FBS DMEM: F12|medium (without G418). Cells were seeded and simultaneously transfected with siRNA using Lipofectamine RNAiMax. Cells were seeded at a density of 225,000 cells per well into a 96-well plate, with 135 μl culture medium per well. Cells were incubated in a 5% CO₂, 37° C. incubator for 3 days. Collected cell supernatants were assayed for HBsAg by ELISA. Simultaneously, cell viability was detected by CellTiter-Glo.

ELISA is briefly described as follows: 50 μl of sample and standard were added to the reaction plate wells, respectively, then 50 μl of enzyme conjugate was added to each well, shaken to mix, incubated at 37° C. for 60 minutes, then washed 5 times with wash buffer, then 50 μl of luminescent substrate was added to each well, mixed, reacted at room temperature while protected from light for 10 minutes, and finally chemiluminescence intensity was detected with a microplate reader.

CellTiter-Glo is briefly described as follows: 100 μl of CTG was added per well, reacted while protected from light for 10 min. Chemiluminescence intensity was detected with a microplate reader.

The antisense oligonucleotides used are described in Table 3.

TABLE 3
Description of oligonucleotides used in Example 2

| | | | S1-S2-S3-S4-S5 |
|---|---|---|---|
| | | | in Gap |
| Oligo- | | | modification |
| nucleotides | Sequence | SEQ ID | patterns |
| PA0088 | GCAGAGGTGAAGCGAAGTGC | SEQ ID NO. 2 | 4-5-1-6-4 |
| PA0089 | GCAGAGGTGAAGCGAAGTGC | SEQ ID NO. 2 | 3-6-1-7-3 |
| PA0114 | GCAGAGGTGAAGCGAAGTGC | SEQ ID NO. 2 | 2-7-1-8-2 |
| PA0090 | GCAGAGGTGAAGCGAAGTGC | SEQ ID NO. 2 | 4-5-2-5-4 |
| PA0115 | GCAGAGGTGAAGCGAAGTGC | SEQ ID NO. 2 | 4-4-2-6-4 |
| PA0091 | GCAGAGGTGAAGCGAAGTGC | SEQ ID NO. 2 | 3-6-2-6-3 |
| PA0116 | GCAGAGGTGAAGCGAAGTGC | SEQ ID NO. 2 | 3-5-2-7-3 |
| PA0092 | GCAGAGGTGAAGCGAAGTGC | SEQ ID NO. 2 | 4-4-3-5-4 |
| PA0093 | GCAGAGGTGAAGCGAAGTGC | SEQ ID NO. 2 | 3-5-3-6-3 |
| PA0094 | GCAGAGGTGAAGCGAAGTGC | SEQ ID NO. 2 | 4-4-4-4-4 |
| PA0095 | GCAGAGGTGAAGCGAAGTGC | SEQ ID NO. 2 | 3-5-4-5-3 |
| PA0096 | GTGCAGAGGTGAAGCGAAGT | SEQ ID NO. 3 | 4-5-1-6-4 |
| PA0097 | GTGCAGAGGTGAAGCGAAGT | SEQ ID NO. 3 | 3-6-1-7-3 |
| PA0098 | GTGCAGAGGTGAAGCGAAGT | SEQ ID NO. 3 | 4-5-2-5-4 |
| PA0099 | GTGCAGAGGTGAAGCGAAGT | SEQ ID NO. 3 | 3-6-2-6-3 |
| PA0100 | GTGCAGAGGTGAAGCGAAGT | SEQ ID NO. 3 | 4-4-3-5-4 |
| PA0101 | GTGCAGAGGTGAAGCGAAGT | SEQ ID NO. 3 | 3-5-3-6-3 |
| PA0102 | GTGCAGAGGTGAAGCGAAGT | SEQ ID NO. 3 | 4-4-4-4-4 |
| PA0103 | GTGCAGAGGTGAAGCGAAGT | SEQ ID NO. 3 | 3-5-4-5-3 |
| PA0104 | AGAGGTGAAGCGAAGTGCAC | SEQ ID NO. 4 | 4-5-1-6-4 |
| PA0105 | AGAGGTGAAGCGAAGTGCAC | SEQ ID NO. 4 | 3-6-1-7-3 |
| PA0106 | AGAGGTGAAGCGAAGTGCAC | SEQ ID NO. 4 | 4-5-2-5-4 |
| PA0107 | AGAGGTGAAGCGAAGTGCAC | SEQ ID NO. 4 | 3-6-2-6-3 |
| PA0108 | AGAGGTGAAGCGAAGTGCAC | SEQ ID NO. 4 | 4-4-3-5-4 |
| PA0109 | AGAGGTGAAGCGAAGTGCAC | SEQ ID NO. 4 | 3-5-3-6-3 |
| PA0110 | AGAGGTGAAGCGAAGTGCAC | SEQ ID NO. 4 | 4-4-4-4-4 |
| PA0111 | AGAGGTGAAGCGAAGTGCAC | SEQ ID NO. 4 | 3-5-4-5-3 |
| Bepirovirsen | GCAGAGGTGAAGCGAAGTGC | SEQ ID NO. 2 | 5-10-5 |

A/T/G represent conventional unmodified deoxyribonucleotide residues and C represents a 5-methyl modified deoxyribonucleotide residue; A/T/G/C represent 2′-O-methoxyethyl modified nucleoside bases; and the internucleotide linkages of each oligonucleotide are all phosphorothioate internucleotide linkages.

The formula for calculating the relative inhibition rate of HBsAg expression/secretion by transfected antisense oligonucleotides in HepG2.2.1.5 cells is:

Relative Inhibition Rate=100%*[1−(HBsAg concentration of treatment group/HBsAg concentration of blank control group)].

The relative inhibition rates for each antisense oligonucleotide treatment group are shown in Table 4.

TABLE 4
Relative inhibition rate of HBsAg expression/secretion
by oligonucleotides in Example 2 in HepG2.2.1.5 cells

HBsAg Relative inhibition rate (%)
Average of Two Experiments

| Oligonucleotides | 10 nM | 30 nM |
|---|---|---|
| PA0088 | 66.77 ± 1.69 | 93.50 ± 0.63 |
| PA0089 | 56.37 ± 0.41 | 86.00 ± 4.04 |
| PA0114 | 25.43 ± 15.99 | 71.74 ± 5.57 |
| PA0090 | 36.93 ± 5.72 | 80.28 ± 6.79 |
| PA0115 | 46.48 ± 11.01 | 82.77 ± 6.02 |
| PA0091 | 38.74 ± 5.45 | 75.15 ± 7.36 |
| PA0116 | 21.56 ± 6.37 | 74.20 ± 4.03 |
| PA0092 | 27.65 ± 6.22 | 61.40 ± 9.27 |
| PA0093 | 39.60 ± 2.11 | 75.47 ± 7.35 |
| PA0094 | 39.62 ± 3.24 | 76.24 ± 5.81 |
| PA0095 | 28.20 ± 4.67 | 60.73 ± 6.24 |
| PA0096 | 21.25 ± 7.31 | 60.35 ± 0.97 |
| PA0097 | 21.38 ± 8.36 | 55.78 ± 9.63 |
| PA0098 | 17.25 ± 3.16 | 47.28 ± 6.24 |
| PA0099 | 13.17 ± 5.59 | 40.91 ± 1.54 |
| PA0100 | 20.36 ± 0.87 | 47.22 ± 6.77 |
| PA0101 | 14.13 ± 5.53 | 49.04 ± 4.87 |
| PA0102 | 16.21 ± 4.08 | 44.53 ± 6.94 |
| PA0103 | 19.95 ± 6.69 | 45.27 ± 5.32 |
| PA0104 | 51.74 ± 4.52 | 84.00 ± 2.44 |
| PA0105 | 24.82 ± 6.73 | 61.12 ± 4.41 |
| PA0106 | 19.13 ± 0.58 | 51.16 ± 5.22 |
| PA0107 | 28.70 ± 0.58 | 64.49 ± 4.72 |
| PA0108 | 27.34 ± 1.52 | 59.84 ± 6.76 |
| PA0109 | 40.41 ± 1.76 | 74.17 ± 1.48 |
| PA0110 | 24.23 ± 1.03 | 51.45 ± 4.27 |
| PA0111 | 33.04 ± 3.35 | 64.08 ± 2.60 |
| Bepirovirsen | 34.78 ± 2.26 | 71.07 ± 0.59 |

From Table 4 and FIG. 2, it can be seen that compared to the single-gap modified antisense oligonucleotide Bepirovirsen, which is in Phase III clinical trials, adding double-gap modified antisense oligonucleotides helps improve the relative inhibition rate of HBsAg. Comprehensive results from the two experiments show that the weighted average of PA0088 and PA0089 is significantly better than that of the single-gap modified antisense oligonucleotide.

### Example 3 EC₅₀ Values of Antisense Oligonucleotides with Different Gap Modification Patterns in HepG2.2.15 Cell Line

The HepG2.2.1.5 cell line stably expresses and replicates HBV virus and secretes HBV viral particles, HBsAg, and HBeAg into the cell supernatant. HepG2.2.1.5 cells were cultured in DMEM: F12|medium (Hyclone) containing 10% FBS (ExCell Bio) and 400 μg/ml G418, and used after three passages.

Day 0: siRNAs were diluted in PBS to concentrations of 100 nM, 33.33 nM, 11.11 nM, 3.70 nM, 1.23 nM, 0.41 nM, 0.14 nM, and 0.05 nM (compound GSK-3228836 concentrations were: 40 nM, 13.33 nM, 4.44 nM, 1.48 nM, 0.49 nM, 0.16 nM, 0.05 nM, and 0.02 nM). HepG2.2.15 cells were taken, washed with DPBS, digested with trypsin, adjusted to appropriate density with 10% FBS DMEM: F12|medium (without G418). Cells were seeded and simultaneously transfected with siRNA using Lipofectamine RNAiMax. Cells were seeded at a density of 225,000 cells per well into a 96-well plate, with 135 μl culture medium per well. Cells were incubated in a 5% CO₂, 37° C. incubator for 3 days. Collected cell supernatants were assayed for HBsAg by ELISA. Simultaneously, cell viability was detected by CellTiter-Glo.

ELISA is briefly described as follows: 50 μl of sample and standard were added to the reaction plate wells, respectively, then 50 μl of enzyme conjugate was added to each well, shaken to mix, incubated at 37° C. for 60 minutes, then washed 5 times with wash buffer, then 50 μl of luminescent substrate was added to each well, mixed, reacted at room temperature while protected from light for 10 minutes, and finally chemiluminescence intensity was detected with a microplate reader.

CellTiter-Glo is briefly described as follows: 100 μl of CTG was added per well, reacted while protected from light for 10 min. Chemiluminescence intensity was detected with a microplate reader.

The antisense oligonucleotides used are described in Table 5.

TABLE 5
Description of oligonucleotides used in Example 3

| | | | P1-S1-P2-S2-P3 |
|---|---|---|---|
| | | | in Gap |
| Oligo- | | | modification |
| nucleotides | Sequence | SEQ ID | patterns |
| PA0088 | GCAGAGGTGAAGCGAAGTGC | SEQ ID NO. 2 | 4-5-1-6-4 |
| PA0089 | GCAGAGGTGAAGCGAAGTGC | SEQ ID NO. 2 | 3-6-1-7-3 |
| PA0090 | GCAGAGGTGAAGCGAAGTGC | SEQ ID NO. 2 | 4-5-2-5-4 |
| PA0091 | GCAGAGGTGAAGCGAAGTGC | SEQ ID NO. 2 | 3-6-2-6-3 |
| PA0093 | GCAGAGGTGAAGCGAAGTGC | SEQ ID NO. 2 | 3-5-3-6-3 |
| PA0094 | GCAGAGGTGAAGCGAAGTGC | SEQ ID NO. 2 | 4-4-4-4-4 |
| PA0104 | AGAGGTGAAGCGAAGTGCAC | SEQ ID NO. 4 | 4-5-1-6-4 |
| PA0109 | AGAGGTGAAGCGAAGTGCAC | SEQ ID NO. 4 | 3-5-3-6-3 |

A/T/G represent conventional unmodified deoxyribonucleotide residues and C represents a 5-methyl modified deoxyribonucleotide residue; A/T/G/C represent 2′-O-methoxyethyl modified nucleoside bases; and the internucleotide linkages of each oligonucleotide are all phosphorothioate internucleotide linkages.

The formula for calculating the relative inhibition rate of HBsAg expression/secretion by transfected antisense oligonucleotides in HepG2.2.1.5 cells is:

Relative Inhibition Rate=100%*[1−(HBsAg concentration of treatment group/HBsAg concentration of blank control group)]

The relative inhibition rates for each antisense oligonucleotide treatment group are shown in Table 6.

TABLE 6
Relative inhibition rate of HBsAg expression/secretion and cell
viability by oligonucleotides in Example 3 in HepG2.2.1.5 cells

| | EC50(nM) | |
|---|---|---|
| Compound | HBsAg | CC50(nM) |
| PA0088 | 1.83 | 18.60 |
| PA0089 | 2.02 | 19.39 |
| PA0090 | 3.04 | 29.42 |
| PA0091 | 3.70 | 45.91 |
| PA0093 | 6.93 | 70.70 |
| PA0094 | 5.37 | 62.12 |
| Bepirovirsen | 19.30 | >40 |

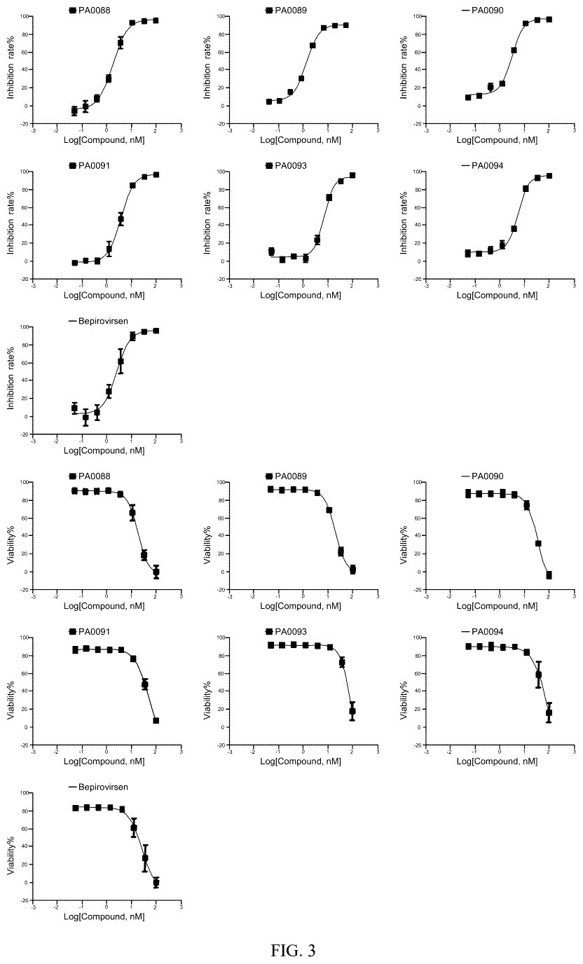

From Table 6 and FIG. 3, it can be seen that double-gap modified compounds help improve the relative inhibition rate of HBsAg. Comprehensive results from the two experiments show that the weighted average of PA0088 and PA0089 is significantly better than that of the single-gap modified antisense oligonucleotide, with EC₅₀ values 10.5 times and 9.6 times lower, respectively, than the control Bepirovirsen (i.e., GSK-3228836).

### Example 4 Antiviral Effects of Double-Gap Antisense Oligonucleotides PA0088, PA0089, and Bepirovirsen in an AAV-HBV Mouse Model

In C57 mice infected with adeno-associated virus carrying 1.3 copies of the HBV genome (AAV-HBV, Wu Jia He) and persistently replicating HBV-DNA and expressing HBV antigens, dose-escalating antisense oligonucleotides PA0020 were administered to evaluate their dose-dependent antiviral activity. The oligonucleotides PA0020 used are shown in Table 7.

| Oligo- | | Oligonucleotides |
|---|---|---|
| nucleotides | SEQ ID | Modification |
| PA0088 | SEQ ID NO. 2 | GCAGAGGTGAAGCGAAGTGC |
| PA0089 | SEQ ID NO. 2 | GCAGAGGTGAAGCGAAGTGC |
| Bepirovirsen | SEQ ID NO. 2 | GCAGAGGTGAAGCGAAGTGC |

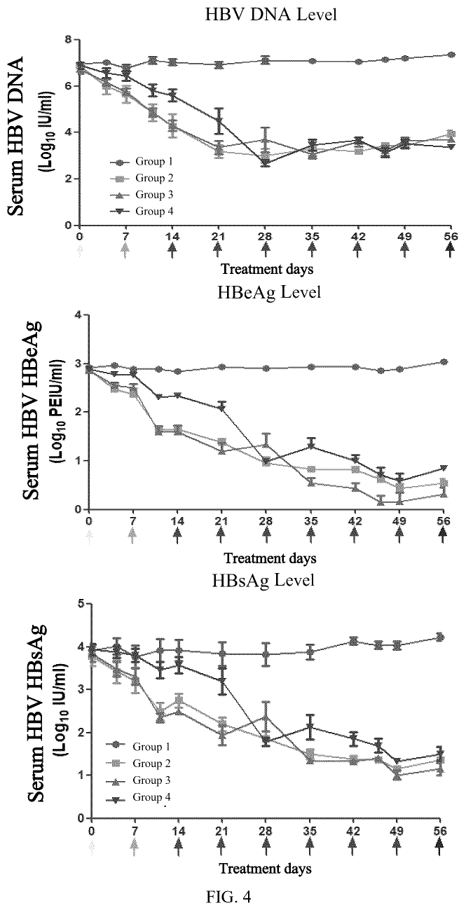

Male C57BL/6 mice were injected via tail vein with 5×10¹⁰ particles of rAAV8-1.3HBV (Wu Jia He) to establish a persistent hepatitis B infection mouse model. After confirming stable HBV viral replication, mice were randomly divided into 4 groups by body weight (5 mice per group). Group 1 was the control group (Vehicle), gavaged daily with physiological saline. Groups 2 to 4 received intraperitoneal injections of Bepirovirsen, PA0088, and PA0089, respectively, at a frequency of once per week, with doses as followss: 30 mg/kg, 60 mg/kg, 60 mg/kg, 90 mg/kg*5. Blood was collected 1-2 times per week. Serum hepatitis B viral nucleic acid (HBV-DNA) load was analyzed by qPCR, and serum HBsAg and HBeAg concentrations were analyzed by ELISA, with curves plotted in FIG. 4.

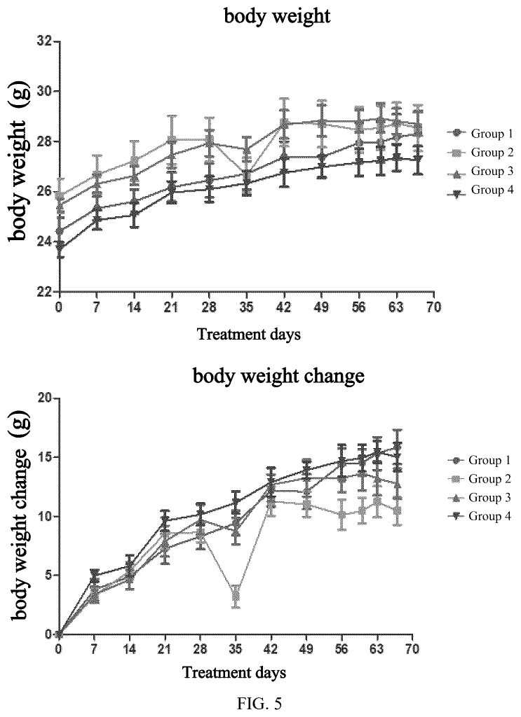

Experimental results as shown in FIG. 4 indicate that: HBsAg and HBV-DNA in the animal serum of the control groups showed stable fluctuations; HBV-DNA in the serum of the Bepirovirsen, PA0088, and PA0089 groups continuously decreased, with a reduction of >3 log₁₀ compared to day 0 at the end of dosing, i.e., a reduction >99.9%. HBsAg and HBeAg in the serum of the Bepirovirsen, PA0088, and PA0089 groups also continuously decreased, with a reduction of >2 log₁₀ compared to day 0 at the end of dosing, with PA0088 showing a slightly greater reduction than the control drug Bepirovirsen. Therefore, PA0088 and PA0089 not only reduce HBV-DNA but also reduce surface antigen, offering hope in the future to achieve functional cure of hepatitis B clinically. Furthermore, as shown in FIG. 5, body weight of all groups of animals increased continuously. The control group Bepirovirsen showed no further weight gain after 42 days of dosing, while PA0088 and PA0089 groups gained more weight, indicating that PA0088 and PA0089 have higher safety than Bepirovirsen.

In particular, the cytosine base in CpG sequences underwent further methylation modification, i.e., cytosine was replaced with 2′-O-methoxyethyl-5-methylcytosine, to reduce its potential to activate Toll-like receptors (TLRs), especially TLR9, thereby minimizing its risk of immune off-target toxicity.

What is claimed is:

1. A compound, or a pharmaceutically acceptable salt thereof, wherein the compound is selected from the following table:

| Oligo- | | |
|---|---|---|
| nucleotides | Sequence | SEQ ID |
| PA0088 | GCAGAGGTGAAGCGAAGTGC | SEQ ID NO. 2 |
| PA0089 | GCAGAGGTGAAGCGAAGTGC | SEQ ID NO. 2 |
| PA0090 | GCAGAGGTGAAGCGAAGTGC | SEQ ID NO. 2 |
| PA0115 | GCAGAGGTGAAGCGAAGTGC | SEQ ID NO. 2 |
| PA0091 | GCAGAGGTGAAGCGAAGTGC | SEQ ID NO. 2 |
| PA0093 | GCAGAGGTGAAGCGAAGTGC | SEQ ID NO. 2 |
| PA0094 | GCAGAGGTGAAGCGAAGTGC | SEQ ID NO. 2 |

in the above table, A/T/G represent conventional unmodified deoxyribonucleotide residues, and C represents a 5-methyl modified deoxyribonucleotide residue; A/T/G/C represent 2′-O-methoxyethyl modified nucleotide bases; and the internucleoside linkages of each oligonucleotide are phosphorothioate internucleoside linkages.

2. The compound of claim 1, wherein the compound is selected from the group consisting of: PA0088, PA0089, PA0090, PA0091, PA0093, and PA0094.

3. The compound of claim 1, wherein the compound is selected from the group consisting of: PA0089, PA0090, PA0091, PA0093, and PA0094.

4. The compound of claim 1, wherein the compound is selected from the group consisting of: PA0089, PA0093, and PA0094.

5. The compound of claim 1, wherein the compound is selected from the group consisting of: PA0089 and PA0093.

6. A pharmaceutical composition, comprising the compound of claim 1, or a pharmaceutically acceptable salt thereof, and a pharmaceutically acceptable carrier.

7. An in vitro non-therapeutic method for regulating HBV DNA/RNA, HBsAg and/or HBeAg expression, the method comprising a step of: contacting a subject with the compound of claim 1, or a pharmaceutically acceptable salt thereof, thereby regulating HBV DNA/RNA, HBsAg and/or HBeAg expression.

8. The method of claim 7, wherein the subject is a cell.


SEQUENCE LISTING (6 sequences)

SEQ ID NO: 1 - DNA, 3182 bp, Hepatitis B virus
  source 1..3182: mol_type = genomic DNA
ttccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtggaa

SEQ ID NO: 2 - DNA, 20 bp, synthetic construct
  source 1..20: mol_type = other DNA
gcagaggtgaagcgaagtgc

SEQ ID NO: 3 - DNA, 20 bp, synthetic construct
  source 1..20: mol_type = other DNA
gtgcagaggtgaagcgaagt

SEQ ID NO: 4 - DNA, 20 bp, synthetic construct
  source 1..20: mol_type = other DNA
agaggtgaagcgaagtgcac

SEQ ID NO: 5 - DNA, 20 bp, synthetic construct
  source 1..20: mol_type = other DNA
gtgaagcgaagtgcacacgg

SEQ ID NO: 6 - DNA, 46 bp, synthetic construct
  source 1..46: mol_type = other DNA
ctgccggaccgtgtgcacttcgcttcacctctgcacgtcgcatgga